(19) United States
(12) Patent Application Publication
(10) Pub. No.: US 2014/0074099 A1
(43) Pub. Date: Mar. 13, 2014

(54) SURGICAL GUIDES AND METHODS FOR MANUFACTURING THEREOF

(71) Applicants: Lara Vigneron, Liege (BE); Louis James Keppler, Leuven (BE); Claudio Fadri Dora, ZH Erlenbach (CH); Stephan Herrman Wirth, Pfaeffikon (CH); Elke Antonia Maria Giets, Nossegem (BE)

(72) Inventors: Lara Vigneron, Liege (BE); Louis James Keppler, Leuven (BE); Claudio Fadri Dora, ZH Erlenbach (CH); Stephan Herrman Wirth, Pfaeffikon (CH); Elke Antonia Maria Giets, Nossegem (BE)

(73) Assignees: University of Zurich, Zurich (CH); MATERIALISE N.V., Leuven (BE)

(21) Appl. No.: 14/079,985

(22) Filed: Nov. 14, 2013

Related U.S. Application Data
(63) Continuation-in-part of application No. PCT/EP2012/059168, filed on May 16, 2012.
(60) Provisional application No. 61/486,357, filed on May 16, 2011.

(30) Foreign Application Priority Data
May 16, 2011 (GB) 1108078.5

(51) Int. Cl. A61B 17/15; A61B 17/17

(52) U.S. Cl. CPC A61B 17/15; A61B 17/17
USPC 606/87; 29/592

(57) ABSTRACT

The present invention relates to surgical guides, and methods for their manufacture. The invention provides surgical guides comprising at least one alignment element and at least two guiding elements. The alignment elements can constrain the guiding elements into an initial position which guides a surgical operation, and optionally at least one target position different from the initial position.

### CROSS-REFERENCE TO RELATED APPLICATIONS

This application is a continuation-in-part of and claims priority of PCT International Patent Application No. PCT/EP2012/059168, filed May 16, 2012, which designates the United States of America and which claims priority to Great Britain Patent Application No. 1108078.5, filed May 16, 2011 and U.S. Provisional Patent Application No. 61/486,357, filed May 16, 2011, the contents of which are incorporated herein by reference in their entirety.

### FIELD OF THE INVENTION

The present invention relates to surgical guides, and methods for the manufacture thereof. The present invention also relates to methods for performing a surgical procedure on an anatomical part. The surgical guides according to the invention have been developed for surgical procedures that require repositioning of anatomical parts, but may further guide surgical operations such as cutting, drilling, screwing, reshaping, reaming and implant positioning.

### BACKGROUND

The success or failure of various surgical procedures depends on the accurate repositioning of a certain anatomical part. For example, accurate repositioning is essential in spinal column surgery (e.g. arthrodesis), and osteotomies.

As an example, Periacetabular Osteotomy (PAO) is a surgical treatment for acetabular dysplasia (a condition defined by inadequate development of an individual's acetabulum) that preserves and enhances the patient's own hip joint rather than replacing it with an artificial part. The PAO cuts the bone around the acetabulum that joins the acetabulum to the pelvis. Once the acetabulum is detached from the rest of the pelvis by a series of carefully controlled cuts, it is rotated to a position of ideal coverage as dictated by the specific acetabulum's unique anatomy. PAO thereby reorients the acetabulum by changing its rotational position. Individual cases of dysplasia however present with their own unique deficiencies and the PAO must often be tailored to solve these unique problems. The osteotomy and reorientation of the acetabulum to its optimal position may be planned based on 2D x-ray radiographs or on 3D CT/MRI, and the preoperative plan can be correlated with intraoperative x-ray imaging to aid the surgeon in the search of the correct position.

Various patient-specific surgical templates and guides are known in the art. Such guides are used to accurately place pins, guide bone cuts or insert implants during orthopedic procedures, and can be developed using commercially available software. The guides can be made from a pre-operative plan formed from an MRI or CT scan of the patient.

US patent application 2008/0087829 (Metzger et al.) describes a patient-specific guiding system for guiding an instrument relative to a portion of an anatomical feature of a patient, including two portions having a patient-specific inner surface that conforms to an anatomical feature. The portions are removably connected to each other, while the first portion includes a guide surface. However, such guide only guides an osteotomy, and not the subsequent repositioning of the bone.

The provision of specific surgical guides implies that the accurate repositioning of the bone requires the time-consuming application of a different guide for the repositioning. Many prior art positioning guides are relatively bulky, which causes a reduced visibility of the bone being worked on. Yet other guides guide the repositioning via screw holes, which is cumbersome in certain procedures.

Therefore there is still a need for guides which mitigate at least one of the problems stated above.

### SUMMARY OF THE INVENTION

The application discloses surgical guiding instruments for positioning on one or more anatomical parts, which help ensure an accurate execution of a surgical operation. More particularly, the application provides surgical guiding systems which provide accurate guidance of one or more surgical tools and optionally guidance for repositioning of anatomical parts or pieces of an anatomical part.

In a first aspect, the surgical guide systems are provided for positioning on one or more anatomical parts. The surgical guide systems comprise one or more independent alignment elements, and at least two patient-specific guiding elements. The guiding elements each have an anatomy engagement surface which anatomically matches (part of) the surface of the anatomical part(s), and each contain one or more coupling features which allow a removable coupling of two or more of guiding elements to the alignment element(s) which serve as a bridge. The coupling features allow the coupling of two or more of the guiding elements to the same alignment element(s) in at least a target position and/or an initial position different from said target position. More particularly, a combination of at least one alignment element and at least two coupling features (one for each guiding element) can allow the coupling of the guiding elements in the target position and/or the initial position.

In certain embodiments, the coupling features allow the coupling of two or more of the guiding elements to the same alignment element(s) in at least an initial position and optionally a target position different from said target position. In particular embodiments, the coupling features allow the coupling of two or more of the guiding elements to the same alignment element(s) in at least a target position and optionally an initial position different from said target position.

In certain embodiments, the guiding elements are coupled to each other in said initial position by one or more fixed connectors. Thus, whereas the coupling of the guiding elements in the target position is typically ensured by coupling the guiding elements to the same alignment element(s) via the coupling features provided on the guiding elements, the coupling of the guiding elements in the initial position can be ensured via one or more fixed connectors. In further embodiments, the guiding elements can be manufactured as a single part.

In particular embodiments, the coupling of the guiding elements in both the initial position and the target position can be ensured via the alignment element(s) and coupling features. More particularly, the combination of different coupling features and/or alignment elements further allows the coupling of the guiding elements to the alignment element(s) in at least one target position different from the initial position. In particular embodiments the surgical guiding systems of the present invention comprise two or more alignment element or at least one adjustable alignment element.

In particular embodiments, the combination of two or more of the guiding elements forms a guiding feature for a surgical tool when they are coupled in the initial position. In particular embodiments, this guiding feature is a cutting slot for an osteotomy.

In particular embodiments, the coupling features comprise elements or combinations of elements selected from the group consisting of interlocking features, a snap-fit system, a dovetail system, a pinned system and a magnetic system. Typically the coupling features are such that they allow an easy interchange of the guiding elements to a different position during the surgical intervention. In particular embodiments, the coupling features are integrated in the guiding elements and/or the alignment elements. More particularly, the coupling features include a complementary shape in the alignment element and the guiding element.

In certain embodiments, at least one of the coupling features allows visual alignment of one or more alignment elements with one or more of the guiding elements.

In particular embodiments, at least one alignment element is patient-specific. In certain embodiments, at least one alignment element contains an anatomy engagement surface which anatomically matches (part of) the surface of the anatomical part(s). In certain embodiments, at least one of the coupling features is patient-specific and/or located on a patient-specific position on at least one of the guiding elements.

In particular embodiments, at least one of the guiding elements and/or alignment element(s) further contains at least one element selected from the group consisting of a drill guide, a screw hole and a cutting slot. In further embodiments, the drill guide contains a drill stop.

In particular embodiments, at least one alignment element or coupling feature is adjustable. In certain embodiments, at least one alignment element comprises fixation features, which allow for fixation of this alignment element to an anatomical part or parts.

In particular embodiments, the anatomical part(s) is a pelvic bone, or one or more vertebrae.

A further aspect provide herein are methods for the manufacture of the surgical guides for positioning on anatomical part(s) described hereabove. The methods comprise steps of:
a) obtaining volume information of the anatomical part(s); and
b) planning a surgical operation or operations; and
c) designing a guiding instrument based on the information obtained in steps a) and b). Thereby, the guiding instrument comprises one or more alignment elements, and at least two patient-specific guiding elements. The guiding elements each have an anatomy engagement surface which anatomically matches (part of) the surface of the anatomical part(s), and each contain one or more coupling features which allow a removable coupling of the guiding elements to the alignment element(s). Moreover, the coupling features allow the coupling of the guiding elements to the alignment element(s) in a target position, and/or an initial position; and
d) producing the alignment element(s) and guiding elements based on the design obtained in step c). In particular embodiments, this step is ensured by additive manufacturing.

In a further aspect methods for performing a surgical procedure on one or more anatomical parts are provided. In particular embodiments, the methods are of interest for the repositioning of two parts of a bone. The methods may comprise the steps of:
i) fitting the at least two guiding elements of the surgical guide as described herein to the anatomical part(s) in an initial position; and
ii) coupling the guiding elements with at least one alignment element in said initial position;
iii) performing a surgical operation on said anatomical part; and
iii′) coupling the guiding elements with one or more alignment elements to constrain the guiding elements in a position different from the initial position; and
iv) optionally, performing a further surgical operation.

The surgical guide systems according to the present invention allow for a more accurate and precise performance of surgical operations such as cutting and drilling in comparison with the known surgical guides currently used.

### BRIEF DESCRIPTION OF THE DRAWINGS

The following description of the figures of specific embodiments of the invention is merely exemplary in nature and is not intended to limit the present teachings, their application or uses. Throughout the drawings, corresponding reference numerals indicate like or corresponding parts and features.

FIG. 1A-1C. Surgical guide (1) for positioning on two anatomical parts (17, 18) according to a particular embodiment of the present invention. The surgical guide (1) comprises two guiding elements (2, 3) with coupling features (5, 6), an alignment element (4) with coupling features (5, 6), and fixation features (10). The alignment element (4) and the guiding elements (2, 3) each comprise at least one anatomy engagement surface (7) A: perspective view of a situation wherein the alignment element (4) is not coupled to the guiding elements (2, 3). B: perspective view of a situation wherein the alignment element (4) constrains the guiding elements (2, 3) in a certain position. C: section view of the situation wherein the alignment element (4) constrains the guiding elements (2, 3) in a certain position.

FIG. 2A-2D. Surgical guide (1) for positioning on one anatomical part (17) according to a particular embodiment of the present invention. The surgical guide (1) comprises two guiding elements (2, 3) with coupling features (5, 6), an alignment element (4) with coupling features (5, 6), and fixation features (10). A: In a certain embodiment, in the initial position, the guiding elements (2, 3) form a cutting slot (8). B: according to a certain embodiment, the guiding elements (2, 3) comprise a planar edge (19) which can be used to guide an osteotomy. C: according to a certain embodiment, at least one guiding element (2) comprises a drill guide (9). D: according to a certain embodiment, at least one guiding element (2) comprises a cutting slot (8).

FIG. 3. Alignment element (4) comprising four coupling features (5, 5′, 6, 6′) according to a particular embodiment of the present invention.

FIG. 4A-4B. Surgical guide (1) for positioning on two anatomical parts (17, 18) according to a particular embodiment of the present invention, comprising an alignment element (4) which is a K-wire (11), and guiding elements (2, 3) with coupling features (5, 6). A: in certain embodiments, the coupling features (5, 6) are cylinders (15) with an optional foot (16). B: in certain embodiments, one or more coupling features (5) is a mark (20) on a guiding element (2).

FIG. 5A-5C. Surgical guide (1) for positioning on two anatomical parts (17, 18) according to a particular embodiment of the present invention, wherein the guiding elements (2, 3) are constrained visually. The surgical guide (1) comprises guiding elements (2, 3) with coupling features (5, 6) and an alignment element (4) with coupling features (5, 6). The guiding elements comprise an anatomy engagement surface (7). A: perspective view of a situation wherein the alignment element (4) is not coupled to the guiding elements (2, 3). B: perspective view of a situation wherein the alignment element (4) constrains the guiding elements (2, 3) visually. C: section view of the situation wherein the alignment element (4) constrains the guiding elements (2, 3) visually.

FIG. 6A-6D. Surgical guide (1) for positioning on two anatomical parts (17, 18) with a pinned connection system, according to a particular embodiment of the present invention. The surgical guide (1) comprises two guiding elements (2, 3), coupling features (5, 6), and an alignment element (4) with coupling features (5′, 6′) containing apertures (14) for receiving a pin (13″). The coupling features further comprise pins (13″) A: perspective view of a situation wherein the alignment element (4) is not coupled to the guiding elements (2, 3). B: perspective view of a situation wherein the alignment element (4) constrains the guiding elements (2, 3) in a certain position. C: section view of the situation wherein the alignment element (4) constrains the guiding elements (2, 3) in a certain position. D: detailed illustration of a standard pin (13), a patient-specific pin (13′) with a mark at a patient-specific height, or a patient-specific and anatomically specific pin (13″) with a mark (20) at a patient-specific height and an engagement surface (7).

FIG. 7A-7B. Perspective view (A) and top view (B) of a surgical guide (1) for positioning on two anatomical parts (17, 18) according to a particular embodiment of the present invention, wherein the guiding elements (2, 3) are constrained visually. The surgical guide (1) comprises two guiding elements (2, 3), coupling features (5, 6) and an alignment element (4) with a coupling feature (5′). The coupling features are marks (20).

FIG. 8A-8B′. Adjustable alignment elements (4, 4′) according to a particular embodiment of the present invention. The alignment elements (4, 4′) are coupled to the guiding elements (2, 3) via coupling features (5, 6, 5′, 6′, 6″). A certain rotation of the alignment elements (4, 4′) constrains the guiding elements (2, 3) in a first position (A: perspective view; A′: top view), whereas another rotation of the alignment elements (4, 4′) constrains the guiding elements (2, 3) in another position (B: perspective view; B′: top view).

FIG. 9A-9B. Alignment elements (4) according to particular embodiments of the present invention, comprising a drill guide (9) and coupling features (5, 6). FIG. 9 A (perspective view) and A′ (top view) show an alignment element (4) comprising a means (21) which allows the measurement of a distance. FIG. 9B shows an alignment element (4) comprising springs (23) and a means (22) which allows the measurement of a force.

FIG. 10A-10C. Fixation of a guiding element (2) to an anatomical part (17) according to particular embodiments of the present invention. A: guiding element (2) comprising fixation features (10) positioned on an anatomical part (17) before fixation. B: guiding element (2) comprising fixation features (10) positioned on an anatomical part (17) after fixation with K-wires (11). C: guiding element (2) comprising cylindrical fixation features (10), positioned on an anatomical part (17) after fixation with K-wires (11).

FIG. 11A-11B. Use of an alignment element (4) according to a particular embodiment of the present invention. A: The alignment element (4) is coupled to the guiding elements (2, 3) via coupling features (5, 6). B: The alignment element (4) is coupled to the guiding elements (2, 3) via coupling features (5, 6′).

FIG. 12A-12K. Use of a surgical guide (1) according to a particular embodiment of the present invention, for guiding an osteotomy and repositioning of a pelvic bone (17), comprising two guiding elements (2, 3), alignment elements (4, 4′, 4″, 4″), drill guides (9, 9′, 9″), fixation features (10), planar edges (19, 19′, 19″) and coupling features (5, 6, 5′, 6′, 5″, 6″). Some coupling features (5″) are marks (20). A: pelvic bone (17) showing the osteotomies to be performed (24, 24′, 24″, 24″). B: the guiding elements (2, 3) are positioned on the pelvic bone (17). B′ is enlarged version of B. C: an alignment element (4) constrains the guiding elements (2, 3) to an initial relative position, wherein a cutting slot (8) and a drill guide (9′) are provided. D: fixation of the guiding elements (2, 3) using K-wires (11). E: an insert (25) and a drill guide (9′) guide a drill (26). F: a drill guide (9″) guides a drill (26). G: a protective filler (29) is inserted into drill guide (9′). H: an insert (25) and a drill guide (9) guide a drill (26). I: a surgical instrument (27) is inserted in drill guide (9). J: alignment elements (4′, 4″) constrain the guiding elements (2, 3) to a target position. J′: alignment element (4′″) visually constrains the guiding elements (2, 3) to a target position. K: fixation of the bone parts (18, 18′) in the target position using fixation pins (28).

FIG. 13A-13C. A: particular embodiment of a surgical guide (1) as described herein, positioned on a pelvic bone (17) in an initial position. B: particular embodiment of a surgical guide (1) as described herein, positioned on two parts (18, 18′) of a pelvic bone in a target position. C: two parts (18, 18′) of a pelvic bone after repositioning guided by a surgical guide as described herein.

In the figures, the following numbering is used:

1—surgical guide; 2, 3—guiding element; 4, 4′, 4″, 4′″—alignment element; 5, 5′, 5″, 6, 6′, 6″—coupling feature; 7—engagement surface; 8—cutting slot; 9, 9′, 9″—drill guide; 10—fixation feature; 11—K-wire; 13, 13′, 13″—pin; 14—aperture; 15—cylinder; 16-foot; 17, 18, 18′—anatomical part, bone; 19, 19′, 19″—planar edge; 20—mark; 21—means for measuring a distance; 22—means for measuring a force; 23—spring; 24, 24′, 24″, 24′″—location for osteotomy; 25—insert; 26—drill; 27—surgical instrument; 28-fixation pin; 29—filler; 30—connector; 31—keyhole; 32—key.

### DETAILED DESCRIPTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +1-0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The terms “connection” and “coupling” are used herein to denote the interaction between one or more guiding elements and one or more alignment elements which can involve either a physical attachment between the elements or a relative positioning based on a visual alignment, or both. Where the term “connection” is used, this essentially refers to the presence of a physical feature which ensures, i.e. physically constrains, a specific relative position of the elements. Where the term “coupling” is used, this refers essentially to the presence of features which allows the relative positioning of the elements based on a visual alignment.

All documents cited in the present specification are hereby incorporated by reference in their entirety.

In a first aspect the surgical guide systems are provided for positioning on one or more anatomical parts. More particularly, the present invention provides surgical guiding instruments for guiding surgical operations including, cutting, drilling, screwing, reshaping, reaming, implant positioning, and the like.

The surgical guide systems of the present invention contain at least two guiding elements which are connected to each other in an initial position; and/or can be fitted relative to each other in an initial position and/or target position by way of an independent alignment element which can be connected to the guiding elements by coupling features present on the guiding elements.

The at least two guiding elements envisaged herein comprise at least one surface which matches part of the surface of the anatomical part(s) for which the surgical guide is designed. This ensures a perfect fit of the guiding elements on the surface of the anatomical part(s). The anatomy engagement surfaces aid the surgeon in finding the correct initial position of the guiding elements on the anatomical part(s).

In particular embodiments, surgical guide systems are provided for positioning on one anatomical part (i.e a single bone). Examples of anatomical parts envisaged in this context are provided herein below but include the pelvic bone, etc. More particularly this implies that the two or more guiding elements comprise a surface which matches part of the surface of that anatomical part. In further embodiments, surgical guides are provided for positioning on two or more anatomical parts. In these embodiments, the guiding elements may each contain a surface which matches part of the surface of a different anatomical part. Examples of different anatomical parts envisaged in the context of this invention are the pelvis, femur, spine, humerus, scapula, clavicle, radius, ulna, carpals, metacarpals, phalanges, tibia, fibula, calcaneus, tarsals, metatarsals, patella, rib, sacrum, coccyx, skull, mandible and maxilla,

The surgical guide systems provided herein further comprise one or more independent alignment elements which can be connected to and/or aligned with two or more of the guiding elements, thereby allowing a visual or physical alignment of the guiding elements in specific positions relative to each other. Indeed the guides of the present invention are envisaged for use in surgical interventions which involve the relative movement of different anatomical parts or different sections of one anatomical part. In the guiding systems of the present invention, the guiding elements are thereby maintained on the respective anatomical part or section, while the coupling of the guiding elements in the different positions can be ensured, for example using a combination of fixed (but severable) connectors and one or more combinations of alignment elements and coupling elements; or using combinations of different alignment elements and/or different coupling features. This allows control of the relative movement of the bones and, optionally, the use of the guiding elements in different relative positions.

The coupling or connection of the guiding elements by the alignment element is ensured by coupling features. In particular embodiments, the coupling features allow a removable connection or coupling of at least one alignment element to at least one of the guiding elements. The coupling features can be provided on the guiding elements and/or alignment elements in such a way that the alignment element(s) can constrain the guiding elements in different positions. More particularly, an alignment element can constrain two guiding elements in a certain position (e.g. the initial position or target position) by combining it with two or more coupling features of the guiding element; more specifically by coupling the same alignment to both guiding elements via least one coupling feature per guiding element.

Different types of coupling features are envisaged in the context of the present invention and the nature thereof is in part determined by the nature of the alignment element(s). In particular embodiments the combination of alignment elements and coupling features are designed to allow a physical constraint of the guiding elements in specific positions, such as the initial position and/or a second position also referred to herein as a target position. Additionally or alternatively, the alignment and coupling features provide a visual reference for the positioning of the guiding elements.

Thus, in particular embodiments, the one or more alignment elements can constrain the guiding elements in one or more positions relative to each other which is/are different from the initial position, i.e. the target position(s). This can be ensured in different ways, e.g. by providing alignment elements of different sizes and shapes, by providing alignment elements which are adjustable and/or by providing coupling features on different positions on the guiding elements and/or alignment elements, such that, depending on the coupling features used for positioning the alignment element(s), the relative position of the guiding elements is changed. This will also be explained more in detail herein below.

In particular embodiments, the alignment elements are used to help to ensure that the positioning of the guiding elements on the anatomical part(s) is in accordance with pre-operative planning. More particularly, the combination of coupling features and one or more alignment elements can constrain the positioning of the guiding elements in the initial position. This allows the surgeon to verify the correct initial position of the guiding elements.

Additionally or alternatively, two or more guiding elements can be constrained physically in the initial position by one or more fixed connectors. More particularly, two or more guiding elements may be provided as a single part, wherein the guiding elements are constrained in the desired initial position by fixed connectors extending between the guiding elements. The dimensions of the connectors is preferably such that they allow for a rigid connection between the guiding elements, while still allowing the separation of the guiding elements by breaking and/or cutting the connectors using standard cutting instruments. In preferred embodiments, two or more fixed connectors are used, to obtain an optimal stability of the connection. In certain embodiments, the connectors may have a straight or bent rod-like shape. The use of fixed connectors eliminates the need of an alignment element for constraining the guiding elements in the initial position. However, once cut or broken, these connectors typically do not allow for reconnecting the guiding elements in the initial position. Therefore, it is envisaged that in certain embodiments, one or more alignment element(s) may provide a backup for re-connecting the guiding elements in the initial position. Additionally or alternatively, one or more alignment elements may be used for providing a stronger connection of the guiding elements in the initial position. In cases where the guiding elements are positioned on different anatomical parts which do not have a fixed relative position, e.g. vertebrae, the initial position of the guiding elements may correspond to a certain relative position of the different anatomical parts, for example the relative positions at the moment when a preoperative scan was obtained. In these cases, the connectors and/or alignment elements may be used to ensure the correct relative positioning of the anatomical parts, instead of ensuring the correct positioning of the guiding elements on the anatomical parts. Accordingly, in certain embodiments, the connectors and/or alignment elements are used to help to ensure that the positioning of the anatomical parts is in accordance with pre-operative planning. Once the correct positioning of the guiding elements on the anatomical part(s) is achieved, the guiding elements can be fixed to the anatomical part(s). Also one or more alignment elements may need to be fixed to the anatomical part(s), for example when an alignment element comprises a guiding feature such as a cutting slot, drill guide, etc. The fixation of guiding elements and/or alignment elements is preferably reversible or removable, for example using fixation means such as Kirschner wires, pins, screws, and the like. This will be discussed more in detail below.

Thus, the surgical guide systems envisaged herein comprise (i) one or more independent alignment elements and (ii) at least two patient-specific guiding elements, with an anatomy engagement surface which anatomically matches part of the surface of the one or more anatomical parts, and comprising at least one coupling feature which allows a removable connection or coupling of the one or more alignment elements to at least one of the guiding elements.

As indicated above, the at least two guiding elements of the surgical guides of the present invention are or can be fitted together in an initial position and optionally in a target position. In particular embodiments, in the initial and/or target position, the guiding elements, together form a guiding feature, such as, but not limited to, a cutting slot for a saw or blade for performing an osteotomy, or a drill guide. This is typically ensured by the fitting together of edges (or parts thereof) of the guiding elements. In most particular embodiments, the edges of the guiding elements, when fitted in the initial or target position, form a cutting slot. In particular embodiments, in the initial and/or target position, the guiding elements, together form at least two guiding features, for example a cutting slot and a drill guide.

The surgical guide systems of the present invention may comprise one or more alignment elements which can constrain the guiding elements in one or more target positions, different from the initial position. This implies that, where applicable, the coupling or connection of the guiding elements in the initial position is removed (or broken or cut in case fixed connectors are used), and the guiding elements are moved to a different position relative to each other. By use of a (different) alignment element and(/or different) coupling features, the surgical guide of the invention may guide the repositioning of certain anatomical parts or subparts thereof.

For example, in particular embodiments, the guiding elements may be placed in an initial position on one anatomical part, such as a bone. After one or more cuts have been performed on the bone, which allow the separation of the bone in two parts, it may be possible to reposition the resulting parts of the bone, while the guiding elements are maintained on the surface of each of the bone parts. In further particular embodiments, when the guiding elements are initially positioned on two or more different anatomical parts, e.g. the guiding elements are positioned different vertebrae, it is possible to reposition the two or more anatomical parts while the guiding elements are maintained on the surface of the respective the anatomical parts. Thus, while the guiding elements are maintained in their position on the bone or anatomical parts, their relative position is changed. This target position, which may correspond to the desired final position of the anatomical part(s), can then be obtained, checked and/or maintained using the appropriate combination of alignment element(s) and coupling feature(s). In embodiments wherein the guiding elements are connected via a first combination of alignment element(s) and coupling features in their initial position, the coupling of the guiding elements in the target position can be obtained using a second combination of alignment element(s) and coupling features which is different from that used in the initial position.

Additionally, the guiding elements and/or alignment elements may, in the initial position and/or target position(s) guide surgical specific operations such as cutting, drilling, screwing, reshaping, reaming and implant positioning. To this end the guiding and/or alignment element(s) may comprise dedicated guiding features. Alternatively, as described above, the combination of guiding elements may form a guiding feature (such as a cutting slot) in the initial or target position. In particular embodiments, each position of the guiding elements, i.e. the initial position and each target position, is ensured by different or different combinations of alignment elements and/or coupling features.

In particular embodiments, the guiding elements form in their initial position one or more cutting slots. For example, two or more guiding elements may each comprise a planar edge, which are positioned parallel to each other in the initial position of the guiding elements. If the guiding elements are connected to each other in the initial position by one or more fixed connectors, one or more connectors may bridge one of the cutting slots which are provided by the guiding elements. Thereby, it can be ensured that by performing the osteotomy guided by the cutting slot, also the connector(s) is/are cut, thereby increasing the efficiency with which the surgeon can perform the procedure.

In certain embodiments, different relative positions of the guiding elements can be obtained by using different combinations of the same alignment elements and/or coupling features. For example, two guiding elements each containing one coupling feature, may be constrained to one relative position using one alignment element, and to another relative position using another alignment element. In that case, both positions are obtained using different alignment elements but the same coupling features on the guiding elements. Alternatively, the one alignment element may be used which is an adjustable alignment element, e.g. comprises a system allowing it to extend into a different size. Another example is where the alignment element has a predefined shape, and the coupling features are interlocking elements provided on the alignment element and in different positions on one or more of the guiding elements. Depending on the coupling features used to position the alignment element, the alignment element will constrain the guiding elements in a different position.

In particular embodiments, a first guiding element contains one coupling feature, and a second guiding element contains two coupling features, such that the guiding elements may be constrained to two different positions, with the same alignment element using a different coupling feature on the second guiding element.

In particular embodiments, at least one of the guiding elements comprises more than one coupling feature. When the surgical guide contains more than one alignment element, each of the coupling features may allow connection or coupling of specific alignment elements or of all alignment elements. In this regard it is noted that some coupling features and/or alignment elements may have specific shapes or be marked in order to aid the surgeon to find the right combination of alignment elements and coupling features. Such markings include, but are not limited to letters, numbers, symbols, specific colours, shapes, etc.

It will be understood by the skilled person that numerous combinations of guiding elements, connectors, coupling features and alignment elements can be envisaged. The principle is that the connectors and alignment elements function as a bridge coupling the guiding elements in different positions relative to each other, while each of the guiding elements is maintained in the same position relative to the bone part.

The combination of alignment elements and coupling features according to the present invention constrains at least two guiding elements to a certain relative position and/or indicates a certain relative position of at least two guiding elements.

In particular embodiments, the combination of coupling features and alignment elements constrains the relative position of the guiding elements mechanically. Mechanical constraint of the guiding elements may be provided by the shape and stiffness of an alignment element and the connection between the alignment elements and the guiding elements ensured by the coupling feature(s). For example, mechanical constraint of the guiding elements can be achieved by an alignment element which is connected to one or both guiding elements through interlocking features, such as, but not limited to a dovetail system, pinned system or magnetic system. These interlocking features may also correspond to (releasable) locking mechanisms, such as snap-fit systems. Thus, in particular embodiments, the coupling features comprise interlocking features, such as dovetail connections, pins, magnets, etc. However, the coupling preferably is reversible, more particularly it is desirable that the coupling to the alignment element can be released during the surgical intervention.

The coupling and/or connection of the at least two guiding elements by the alignment element, may be the same or different for each guiding element. However, in particular embodiments, the connection or coupling to the different alignment elements is based on the same system.

Additionally or alternatively, the combination of alignment and coupling features constrains the relative position of the guiding elements visually. In this case, the combination of alignment and coupling features can be used to obtain or check a particular relative position of the guiding elements. For example, the alignment element can be coupled to only one of the guiding elements and its orientation relative to a point on the second guiding element can be used as visual guidance for the position of the second guiding element. This “point” can be a visual coupling feature which can be three-dimensional (e.g. a protrusion on the surface of the guiding element) or a visual mark. In certain embodiments, the relative position of the guiding elements is obtained or checked by aligning the alignment element to the visual coupling feature. In particular embodiments, the visual alignment is ensured by a combination of two or more visual coupling features present on the alignment element and/or the guiding elements, optionally in combination with visual reference points on the anatomical part(s).

In particular embodiments, the coupling features on both the alignment element and the guiding element(s) are visual marks and the initial and/or target position is obtained by aligning the visual marks on the guiding elements and the alignment element. In certain embodiments, the guiding elements and alignment elements can be arranged such that the marks, i.e. the coupling features, form a shape such as, but not limited to a straight line, a rectangle, a square, a circle, etc. Similarly, three-dimensional shapes are envisaged.

In particular embodiments, at least one of the coupling features is a feature which ensures physical connection of the alignment element to a guiding element. Typically, the combination of alignment and coupling features constrains the relative position of the guiding elements both mechanically and visually. In particular embodiments, at least one alignment element is patient-specific. For example, the alignment element may have an adapted shape and/or adapted dimensions which allows for the constraint of the guiding elements in patient-specific positions. One of the advantages of patient-specific alignment elements is that they can be used to achieve a patient-specific relative position of the guiding elements, while making use of coupling features which are standard, i.e. not patient-specific. Moreover, patient-specific alignment elements are usually less bulky than standard alignment elements. Where the dimension of the alignment element is critical to ensure the correct relative position of the guiding elements, the one or more alignment elements are manufactured based on a patient-specific pre-operative planning. For example, in particular embodiments, the alignment element is a bridge which can be connected to the guiding elements by a dovetail connection. Bridges of different length can be provided, based on pre-operative planning of the surgical procedure on the patient, which allow connection of the guiding elements in different relative positions, while using the same or different coupling features (of a dovetail connection) present on the guiding elements.

In particular embodiments, at least one alignment element is specific to the anatomy of the patient. For example, such an alignment element may contain an anatomy engagement surface which is designed so as to engage or mate with the surface of an anatomical part. This engagement surface may be located anywhere on the alignment element. If the alignment element comprises coupling features, one or more of the coupling features may contain an anatomy engagement surface. The anatomy engagement surface helps the surgeon in finding and verifying the correct position of the guiding elements with respect to each other and to the anatomical part(s).

In particular embodiments, at least one alignment element is an adjustable element. For example, the shape and/or the dimensions can be adjusted in such a way that one alignment element allows for the constraint of the guiding elements in different (patient-specific) positions. Adjustable alignment elements allow for real-time adapting of the planning of the surgical operation or operations. For instance, in particular embodiments, an alignment element is provided which is extendible.

In particular embodiments, at least one alignment element is a standard instrument. As indicated above, in these embodiments, the patient specific constraint can be ensured by the position of the coupling features, which allow connection or coupling of the alignment element to one or more of the guiding elements. Thus, in these embodiments, the position of the coupling feature can be patient-specific. Non-limiting examples of standard instruments include Kirschner wires (K-wires), pins, rods, screws, drills and the like. In particular embodiments, the alignment element is a K-wire and the coupling feature is a cylinder attached to each guiding element. In order to determine the correct relative position of the two guiding elements, the K-wire can be slipped into the cylinders. The standard alignment element and the coupling features ensure a physical constraint. In alternative embodiments, the alignment element is a K-wire and the coupling mechanism is a cylinder attached to one guiding element only. When positioning of the K-wire in the cylinder, the orientation of the K-wire can be aligned with a specific point on the second guiding element. In this way the coupling and alignment element ensure a visual constraint (as detailed above).

Thus, in particular embodiments, at least one coupling feature is patient-specific. More particularly a coupling feature can have a patient-specific position and/or orientation on a guiding element. In further particular embodiments, at least one coupling feature has a patient-specific position and/or orientation on an alignment element. An advantage of patient-specific coupling features is that they allow the use of less bulky alignment elements and/or standard alignment elements, i.e. not patient-specific alignment elements. Additionally or alternatively, patient-specific coupling features may be coupling features which contain patient-specific marks, for example a pin containing a mark at a patient-specific height.

In particular embodiments, at least one coupling feature is specific to the anatomy of the patient. For example, such a coupling feature may be a pin which contains an anatomy engagement surface which is designed so as to engage or mate with the surface of an anatomical part. In particular embodiments, this may help the surgeon in finding and verifying the correct position of the guiding elements with respect to each other and to the anatomical part(s).

In particular embodiments, at least one coupling feature is adjustable, i.e. such a coupling feature has an adjustable position and/or orientation. Adjustable coupling features allow for real-time adapting of the planning of the surgical operation or operations.

Thus, it will be clear to the skilled person that different combinations of patient-specific (optionally adjustable and/or anatomically specific) or standard (optionally adjustable) alignment elements and patient-specific (optionally adjustable and/or anatomically specific) or standard (optionally adjustable) coupling features can be envisaged in the context of the present invention to allow visual and/or mechanical alignment of the guiding elements in one or more positions.

In certain embodiments, at least one alignment element further comprises a means which allows the measurement of a physical variable, such as a distance and/or a force. For example, an alignment element may comprise a spring or a digital force sensor. The readout may be digital, on a local display or on an external device, or analog, e.g. via gear dials. With a digital readout, info about the measured physical variable or variables may be stored over time in a memory.

The means which allows the measurement of a physical variable may be patient-specific, or standard, and may be reusable on other alignment elements.

Advantageously, the alignment elements which comprise a means which allows the measurement of a physical variable may also be adjustable. Such alignment elements may further comprise one or more locking features. In this way, the alignment elements can be adjusted to a desired distance or force, and then locked at that distance or force. Suitable locking mechanisms include a locking pin, a latch with different notch distances, or rotating alignment elements (see example 8).

In particular embodiments of the surgical guide systems described herein, one or more of the guiding elements further contains one or more fixation features, which allow for fixation of the guiding element to one or more anatomical parts. In particular embodiments of the surgical guides according to the present invention, one or more of the alignment elements further contains one or more fixation features, which allow for fixation of the alignment element to one or more anatomical parts. Possible fixation features include, but are not limited to apertures, fixation cylinders, locking systems and the like. The fixation features allow fixation of a guiding element and/or alignment element to an anatomical part, for example using pins, plugs, wires, screws or drills. In preferred embodiments, the fixation features allow reversible or removable fixation of the guiding element and/or alignment element, for example using fixation means such as Kirschner wires, pins, or, more particularly screws. In particular embodiments, at least one of the fixation features is threaded. The fixation features may moreover contain features for positioning of a guide for a surgical instrument, such as a drill guide. This allows the drilling of a hole in the anatomical part(s) such that a fixation means can be introduced for fixation of the guiding element.

The guiding elements and/or alignment elements of the surgical guide provided herein may contain at least one element which functions as a guiding feature, such as those selected from the group consisting of a drill guide, and a cutting slot. These features may facilitate the use of surgical instruments on the anatomical part(s). In particular embodiments, these features ensure that the guiding elements and/or alignment elements ensure guidance of one or more osteotomies.

In particular embodiments, the guiding elements and/or alignment elements of the surgical guide according to the present invention may contain at least one flat and/or planar and/or curved edge which forms a plane along which a surgical blade can positioned. Such an edge can be used to guide an osteotomy in the plane of or along the edge.

In particular embodiments, the guiding elements form a first guiding feature for one major surgical step, such as an osteotomy, when placed in the initial position, but additional osteotomies with different orientations are required for the repositioning of the anatomical part. Additional osteotomies in different angles may in particular embodiments be prepared by drilling a hole in the surface of the (first) osteotomy. Such drilling weakens the bone along the osteotomy, which makes it easier for the surgeon to perform that osteotomy.

Thus, in particular embodiments, at least one guiding element or alignment element contains at least one drill guide which provides a guide for drilling within the surface of an osteotomy to be performed. This is ensured by determining the orientation of the drill guide by pre-operative planning. In certain embodiments, the guiding elements form in their initial and/or target position two cutting slots and one drill guide, wherein the drill guide allows for guiding a drill along the intersection of (the planes defined by) the two cutting slots. Drilling along said intersection can weaken the bone at the intersection, thereby facilitating the osteotomies guided by the cutting slots.

In particular embodiments, one or more of the drill guides present on the guiding elements and/or alignment elements according to the invention contains a drill stop. More particularly the drill-stop feature is such that it ensures the stopping of the drill at the intersection with the surface of another (third) osteotomy to be performed. This makes it easier for the surgeon to perform the (second) osteotomy, as it will allow him to stop cutting at the right location and will give some indication whether the surgeon is cutting in the right direction.

In particular embodiments, at least one of the guiding elements and/or alignment elements comprises a feature which guides the use of a surgical instrument such as, but not limited to a reamer, an impactor, a chisel, a saw blade, a bone handle, a needle, a shaver, a burr, and the like.

The surgical guide systems described herein are envisaged to be of particular interest in the guiding, positioning and repositioning of bones and bone parts, and implant preparation. Thus, in particular embodiments, the anatomical part(s) is a bone or bones.

The nature of the bone is not critical to the present invention. However, the invention is of particular interest for bones for which surgery implies repositioning and/or osteotomy. Osteotomy and/or (re)positioning are typically performed to correct a hallux valgus, or to straighten a bone that has healed crookedly following a fracture. It is also used to correct a coxa vara, genu valgum, and genu varum. In particular embodiments, the surgical guides of the present invention are used for positioning on a pelvic bone, an acetabulum (of the hip) for surgeries including periacetabular osteotomy, Bernese pelvic osteotomy, Chiari osteotomy, innominate osteotomy, spherical periacetabular osteotomy, a mandible or maxilla (of the jaw), a femur for surgeries including distal femoral osteotomy, intertrochanteric and transtrochanteric osteotomy, derotational osteotomy, corrective osteotomy, Imhaeuser's osteotomy, one or more vertebrae of the spine for surgeries including spine correction, spine fusion, spine facetectomy, spine laminectomy, spondylolisthesis treatment, the chin or the tibia for surgeries including high tibial osteotomy, carpal bones for surgeries including 3- and 4-corner fusion and schaphoid nonunion treatment. The present invention can be applied both in human and in veterinary medicine.

A further aspect provides methods for the manufacture of the surgical guide systems described herein.

The surgical guides according to the present invention are patient-specific guides, As detailed above, the guiding elements and optionally the alignment elements contain anatomy engagement surfaces which mate with the surface of an anatomical part(s). In addition, in particular embodiments, the size and shape of the guiding elements, alignment element(s) and the position of the coupling features thereon is patient-specific. The generation of patient-specific engagement surfaces and patient-specific elements is done based on pre-operative images of the anatomical part(s) and planning of the surgery. Accordingly, methods for producing the (patient-specific) guiding instruments according to the invention typically comprise the steps of:
a) obtaining volume information of said anatomical part(s);
b) planning a surgical operation or operations;
c) designing a guiding instrument based on the information obtained in steps (a) and (b), said guiding instrument comprising:
   at least one alignment element;
   at least two patient-specific guiding elements, each having a anatomy engagement surface which anatomically matches part of the surface of said anatomical part(s), and each containing one or more coupling features which allows a removable connection or coupling of said alignment element to said guiding elements;
   wherein said coupling features allow the connection or coupling of said guiding elements to said one or more alignment elements in a target position and/or an initial position; and
d) producing said alignment element and said guiding elements based on said design.

In particular embodiments, the guiding instruments of the present invention are further designed such that the coupling features allow the connection or coupling of said guiding elements to said one or more alignment elements in a (target or initial) position whereby in said position a guiding feature for a surgical tool as required by said planning is formed by said guiding elements. Additionally or alternatively, the guiding instruments of the present invention can be designed such that the guiding elements are coupled to each other in an initial position by one or more fixed connectors, whereby in said initial position a guiding feature for a surgical tool as required by said planning is formed by said guiding elements.

The method for manufacturing the guiding instruments according to the invention typically comprise the step of identifying and selecting suitable locations and/or orientation for coupling features, drill guides, screw holes, cutting slots and fixation mechanisms.

The step of obtaining volume information of the anatomical part(s) typically comprises obtaining digital patient-specific image information which can be done by any suitable means known in the art, such as for example a computer tomography (CT) scanner, a magnetic resonance imaging (MRI) scanner, an ultrasound scanner, or a combination of Roentgenograms. A summary of medical imaging has been described in “Fundamentals of Medical imaging”, by P. Suetens, Cambridge University Press, 2002.

In a particular embodiment, Additive Manufacturing (AM) techniques are used for manufacturing the guiding instrument according to the invention, or parts thereof. AM techniques are particularly suitable for the manufacture of patient-specific instruments, for example guiding elements comprising a patient-specific anatomy engagement surface. AM further allows for the manufacture of two or more guiding elements as a single part, wherein the guiding elements are connected via fixed connectors. Additive Manufacturing (AM) can be defined as a group of techniques used to fabricate a tangible model of an object typically using three-dimensional (3-D) computer aided design (CAD) data of the object. Currently, a multitude of Additive Manufacturing techniques is available, including stereolithography, Selective Laser Sintering, Fused Deposition Modeling, foil-based techniques, etc.

Selective laser sintering uses a high power laser or another focused heat source to sinter or weld small particles of plastic, metal, or ceramic powders into a mass representing the 3-dimensional object to be formed.

Fused deposition modeling and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically AM techniques start from a digital representation of the 3-D object to be formed. Generally, the digital representation is sliced into a series of cross-sectional layers which can be overlaid to form the object as a whole. The AM apparatus uses this data for building the object on a layer-by-layer basis. The cross-sectional data representing the layer data of the 3-D object may be generated using a computer system and computer aided design and manufacturing (CAD/CAM) software.

The guiding instruments according to the present invention may be manufactured in different materials. Typically, only materials that are biocompatible (e.g. USP class VI compatible) with the animal or human body are taken into account. Preferably the surgical template is formed from a heat-tolerable material allowing it to tolerate high-temperature sterilization. In the case selective laser sintering is used as an AM technique, the surgical template may be fabricated from a polyamide such as PA 2200 as supplied by EOS, Munich, Germany or any other material known by those skilled in the art may also be used.

A further aspect provides methods for using the surgical guide systems described herein. More particularly, the surgical guides of the present invention are used for the guiding of surgical procedures such as the repositioning of (parts of) one or more anatomical parts and/or performing osteotomies.

In particular embodiments, the present invention provides methods for using the surgical instruments described herein, comprising the steps of:

(i) Fitting at least two guiding elements to one or more anatomical parts. Each of the guiding elements comprises at least one surface which matches part of the surface of the one or more anatomical parts, thus ensuring a perfect fit of the guiding elements on the surface of the one or more anatomical parts. In particular embodiments, the guiding elements are fitted to the one or more anatomical parts, such that a guiding feature, e.g. a cutting slot or a drill guide, is formed by the two guiding elements.
(ii) Using at least one alignment element to verify and/or stabilize the correct relative position of the guiding elements. The correct relative position is determined by preoperative planning. Step (ii) is optional as the perfect fit obtained in step (i) is a good indication that the positioning of the guiding elements on the anatomical part(s) is in accordance with the preoperative planning. However, the use of one or more alignment elements allows for a more accurate positioning of the guiding elements. Furthermore, step (ii) is not needed when the guiding elements are coupled to each other in the initial position by one or more fixed connectors.
(iii) Optionally, fixing the guiding elements to the anatomical part(s), said fixation preferably being reversible or removable, for example using fixation means such as Kirschner wires, pins, screws and the like. In particular embodiments, step (iii) is not present, or optional.
(iv) Optionally, performing at least one surgical operation. In particular embodiments this operation is guided by at least one guiding feature selected from the group consisting of a cutting slot, a drill guide, and a screw hole, said guiding feature being provided on one or both of the guiding elements and/or alignment elements, or provided by the combination of the guiding elements in a certain relative position. Non-limiting examples of surgical operations which may be guided by the guiding feature or features are cutting, drilling, screwing, reshaping, reaming, implant positioning, and the like. In particular embodiments the two guiding elements form a cutting slot. Where the repositioning of the bone parts in two different positions is envisaged, the methods may further comprise one or more of the steps of:
(v) Removing the one or more alignment elements constraining the guiding elements in the initial position and/or cutting the fixed connectors which constrain the guiding elements in the initial position. This step can be ensured before, during or after the surgical operation. This allows the guiding elements to move relative to each other.
(vi) Optionally, using at least one alignment element to constrain the guiding elements to a target position different than the initial position. The relative position of the at least two anatomical parts are thereby fixed into a target position different from the initial position. It is envisaged that more than one target position can be of interest.
(vii) Optionally, performing at least one surgical operation. In particular embodiments this operation is guided by at least one guiding feature selected from the group consisting of a cutting slot, a drill guide, and a screw hole, said guiding feature being provided on one or both of the guiding elements and/or alignment elements, or provided by the combination of the guiding elements in a certain relative position. Non-limiting examples of surgical operations which may be guided by the guiding feature or features are cutting, drilling, screwing, reshaping, reaming, implant positioning, and the like. In particular embodiments the two guiding elements form a cutting slot.

Depending on the purpose of the guiding instrument steps (iv) to (vii) are optional and/or repeated several times. As detailed herein above, in particular embodiments, the guiding instruments are envisaged for repositioning of one or more anatomical parts of parts thereof. In these embodiments, the guiding instrument may or may not additionally serve as a guiding instrument for drilling, osteotomies etc. In further particular embodiments, the guiding instruments of the present invention are envisaged for use in the guiding of an osteotomy and may or may not be used for guiding the respositioning of the bones or bone parts.

In particular embodiments, methods are envisaged which include alternation of steps (i), (ii), (iii), (iv) and/or (v), (vi), (vii) in any order and any number of times necessary to perform a surgical operation or operations. In preferred embodiments, the fixation of a guiding element according to step (iii) only occurs once per guiding element.

The invention will now be illustrated by the following, non-limiting illustrations of particular embodiments of the invention.

### EXAMPLES

### Example 1

### Surgical Guide for Positioning on Two Anatomical Parts

The surgical guides according to the present invention can be designed for positioning on two anatomical parts (17, 18). In this embodiment of the invention, each guiding element (2, 3) is positioned on a different anatomical part. Both guiding elements comprise a coupling feature (5, 6) which match a coupling feature (5, 6) on the alignment element (4), thus allowing the coupling of an alignment element (4) to the guiding elements (2, 3). The guiding elements can be fixed onto the anatomical parts via fixation features (10) which are apertures.

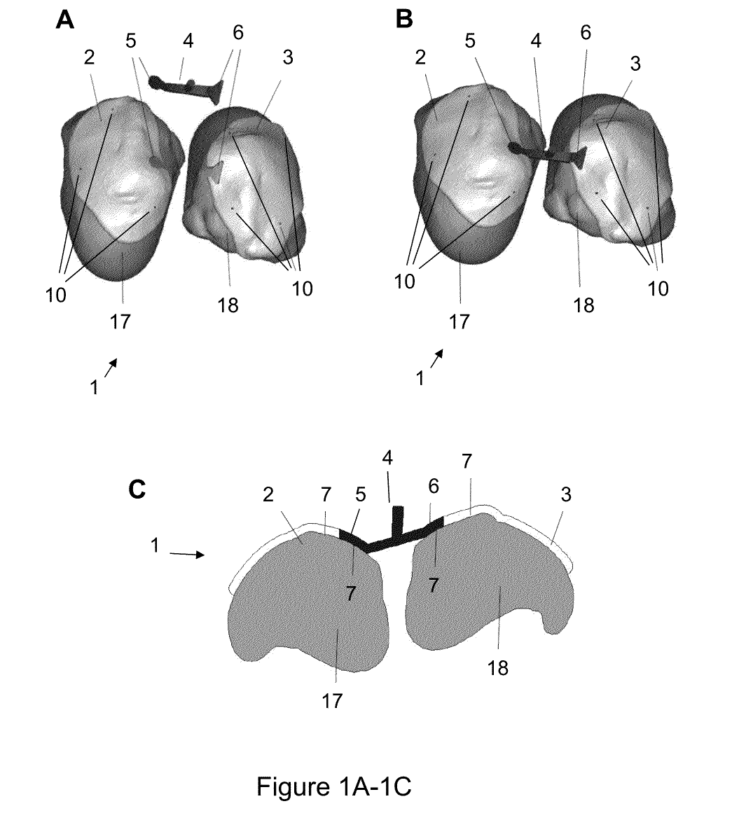

FIG. 1A shows a situation wherein the alignment element (4) is not coupled to the guiding elements (2, 3). In this case, the guiding elements, and consequently also the anatomical parts (17, 18) are not constrained in a specific position.

FIG. 1B shows a situation wherein the alignment element (4) is coupled to the guiding elements (2, 3) via the coupling features (5, 6). The coupling features provide interlocking elements. In this case, the guiding elements, and consequently also the anatomical parts (17, 18) are constrained mechanically in a specific position. The two coupling features (5, 6) of the alignment element (4) have a different shape. Thus, each coupling feature (5, 6) of the alignment element (4) can only couple to specific coupling features (5, 6) on the guiding elements (2, 3), which avoids incorrect coupling of the alignment element (4) to the guiding elements (2, 3).

FIG. 1C shows a section view of the situation wherein the alignment element (4) is coupled to the guiding elements (2, 3) via the coupling features (5, 6). FIG. 1C further shows that not only the guiding elements (2, 3), but also the alignment element (4) may comprise one or more anatomy engagement surfaces (7).

It will be clear to the skilled person that by using more than two guiding elements, guides for the positioning on more than two anatomical parts can be designed.

Similar guides can be designed, wherein the guiding elements are constrained visually. An example of such a guide is discussed in Example 5.

### Example 2

### Surgical Guide for Positioning on One Anatomical Part

The surgical guides according to the present invention can be designed for positioning on one anatomical part (17). In this embodiment of the invention, the guiding element (2, 3) are positioned on the same anatomical part. The guiding elements each comprise a coupling feature (5, 6) which allows the coupling of an alignment element (4) via its coupling features (5, 6) to the guiding elements (2, 3). The guiding elements further comprise fixation features (10) which are apertures.

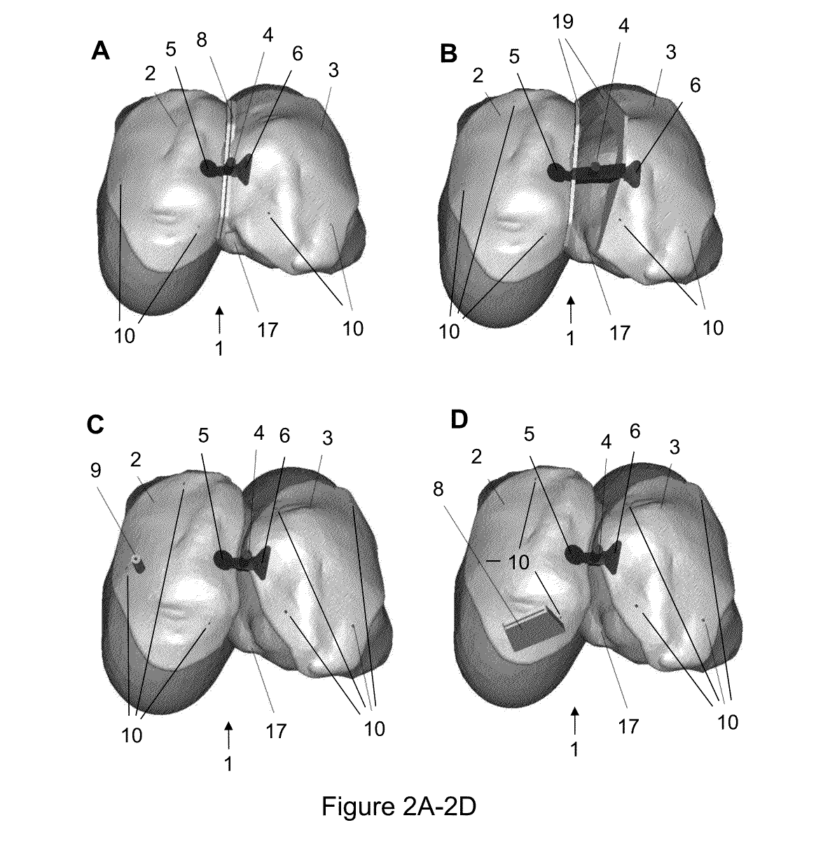

FIG. 2 shows various embodiments of surgical guides positioned on one anatomical part. FIGS. 2A, B, C and D all show two guiding elements (2, 3) positioned on an anatomical part. An alignment element (4) constrains the guiding elements to a certain position, which can be the initial or a target position.

In the guide shown in FIG. 2A, the relative position of the guiding elements (2, 3) provides a cutting slot (8). In the guide shown in FIG. 2B, each guiding element (2, 3) comprises a planar edge (19). Each planar edge can be used to guide an osteotomy. In the guide shown in FIG. 2C, one guiding element (2) comprises a drill guide (9). In the guide shown in FIG. 2D, one guiding element (2) comprises a cutting slot (8).

It will be apparent to the skilled person that the embodiments shown in FIG. 2 may be combined, and that the guiding elements according to the present invention may provide more than one cutting slot in a certain position, or comprise more than one planar edge, drill guide or cutting slot.

### Example 3

### Alignment Element Comprising More than Two Coupling Features

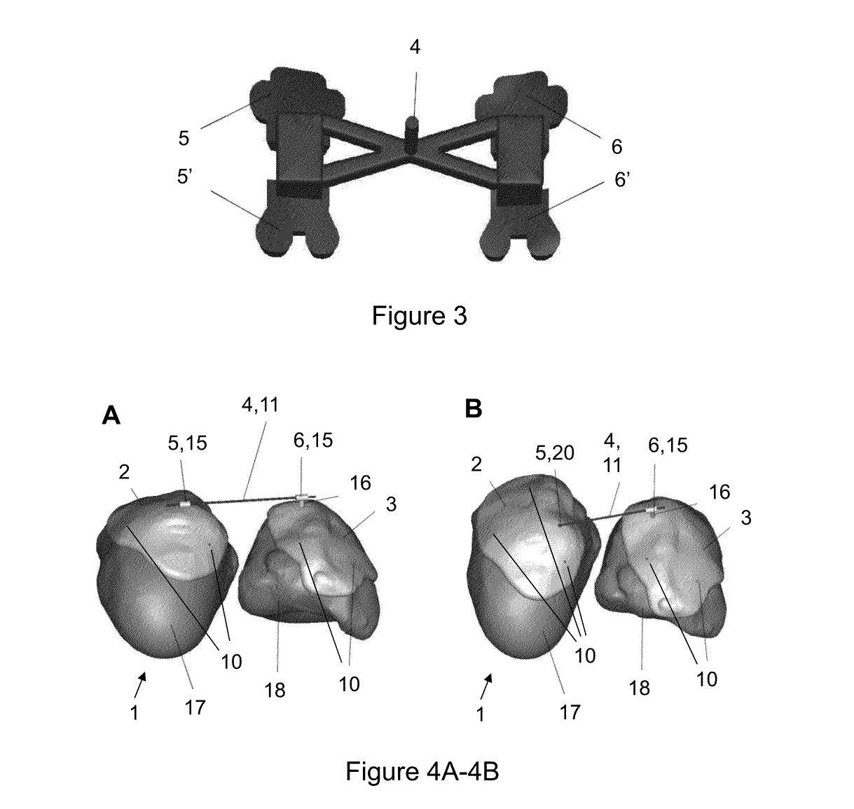

In particular embodiments, the alignment elements according to the present invention comprise a multitude of coupling features. For example, FIG. 3 shows an alignment element (4) comprising four coupling features (5, 5′, 6, 6′). This allows for an improved accuracy and stability during the positioning of the guiding elements. In order to improve the accuracy, the coupling features may also be anatomically specific.

### Example 4

### Cylindrical Coupling Features

This example is shown in FIG. 4. In this embodiment, the coupling features (5, 6) can be cylinders (15) with an optional foot (16), attached to the guiding elements (2, 3). The alignment element (4) is a K-wire (11).

FIG. 4A shows two guiding elements (2, 3) positioned on two anatomical parts (17, 18). The guiding elements can be fixed onto the anatomical parts via fixation features (10) which are apertures. Each guiding element comprises a coupling feature (5, 6) which is a cylinder (15). On one guiding element, the cylinder (15) has a foot (16). Finding the correct relative position of the guiding elements, and thus of the anatomical parts, involves sliding a K-wire (11) into both cylinders (15).

FIG. 4B shows two guiding elements (2, 3) positioned on two anatomical parts (17, 18). One guiding element (3) comprises a coupling feature (6) which is a cylinder (15) on a foot (16), whereas the coupling feature (5) on the other guiding element (2) is a mark (20). Finding the correct relative position of the guiding elements, and thus of the anatomical parts, involves sliding a K-wire (11) into the cylinder (15), thereby letting one end of the K-wire touch the mark (20). Various mechanisms may prevent the translation of the K-wire along the cylinder. For example, the cylinder may be closed at one end, or the K-wire may be fixed using a screw.

### Example 5

### Surgical Guide for Positioning on Two Anatomical Parts

This example is identical to Example 1, except for the coupling features on the guiding elements and alignment elements.

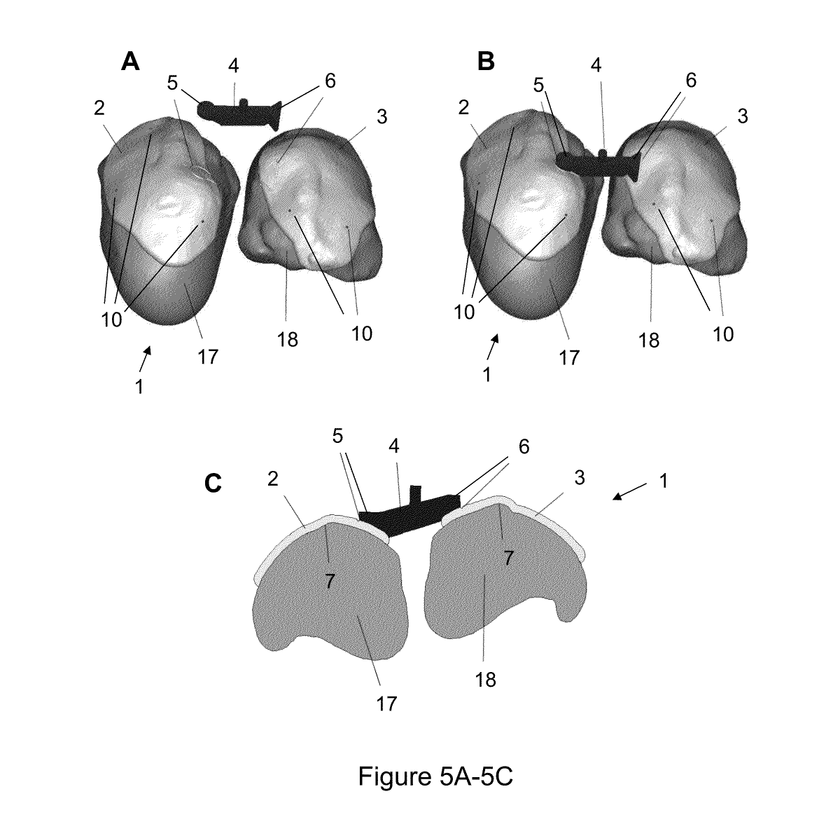

FIG. 5A shows a situation wherein the alignment element (4) is not coupled to the guiding elements (2, 3). In this case, the guiding elements, and consequently also the anatomical parts (17, 18) are not constrained in a specific position.

FIG. 5B shows a situation wherein the alignment element (4) is coupled to the guiding elements (2, 3) via the coupling features (5, 6). The coupling features on the guiding elements are marks which have a shape that follows the contour of the coupling features of alignment element (4). In this case, the guiding elements, and consequently also the anatomical parts (17, 18) are constrained visually in a specific position.

FIG. 1C shows a section view of the situation wherein the alignment element (4) is coupled to the guiding elements (2, 3) via the coupling features (5, 6). FIG. 1C further shows that alignment element (4) does not comprise an anatomy engagement surface, whereas the guiding elements do comprise an anatomy engagement surface (7).

### Example 6

### Pinned Coupling Mechanism

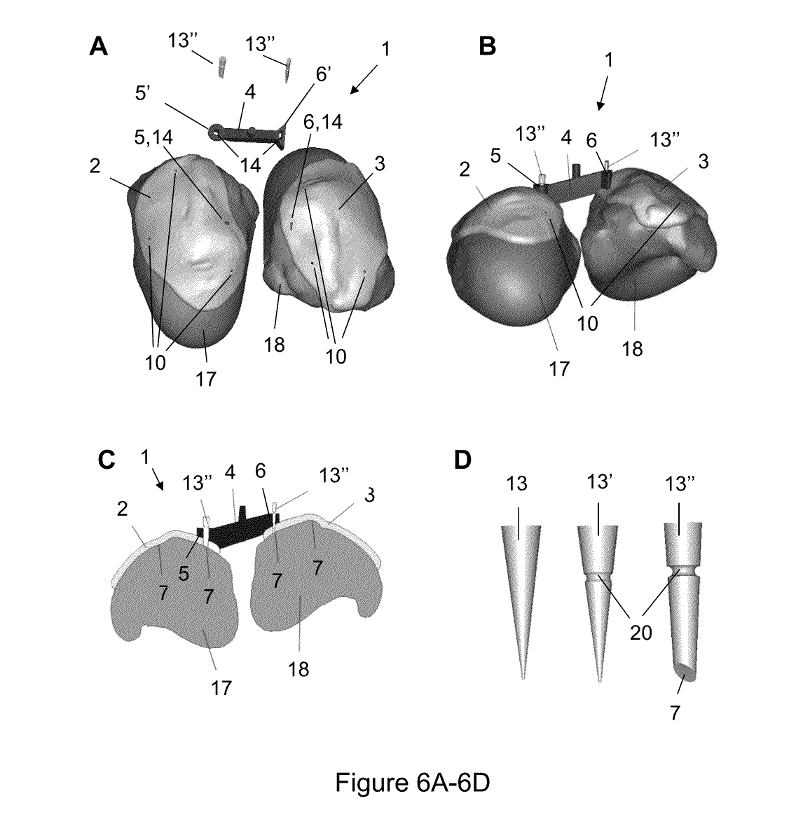

Alignment elements can constrain the guiding elements of the surgical guides according to the present invention to a specific position by means of a pinned system, as shown in FIG. 6A-C. In this embodiment, the coupling features (5, 6) of the guiding elements comprise a pin (13″) and an aperture (14) which can receive a pin. Also the alignment elements contain coupling features (5′, 6′) which comprise an aperture (14) for receiving a pin.

As shown in FIG. 6D, the pin may be a standard pin (13), a patient-specific pin (13′) with a mark (20) at a patient-specific height, or a patient-specific and anatomically specific pin (13″) with a mark at a patient-specific height and an engagement surface (7). Especially if one or more pins are patient-specific or anatomically specific, the apertures (14) and the pins (13, 13′, 13″) may have different shapes, as shown in FIG. 6A. In this way, each pin only matches one aperture (14), which helps in finding the correct positions.

### Example 7

### Visual Constraint

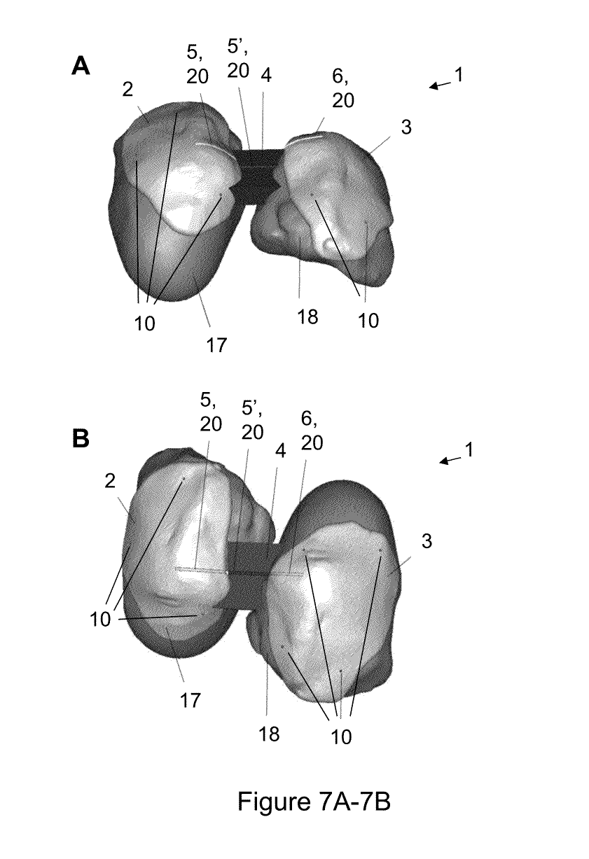

An example of visual constraint of the guiding elements is shown in FIG. 7. The guiding elements (2, 3) are positioned on anatomical parts (17, 18) and both guiding elements contain coupling features (5, 6). The coupling features (5, 6) are line-shaped marks (20). Also alignment element (4) contains a coupling feature (5′) which is a line-shaped mark (20). Visual constraint of the guiding elements (2, 3) is achieved when the alignment element (4) is arranged such that the line-shaped mark on alignment element (4) forms a continuous line with the line-shaped coupling features (5, 6). Alignment element (4) may further contain an engagement surface which matches with the surface of the anatomy, thus offering a more reliable and stable configuration of the guiding elements.

### Example 8

### Adjustable Alignment Elements

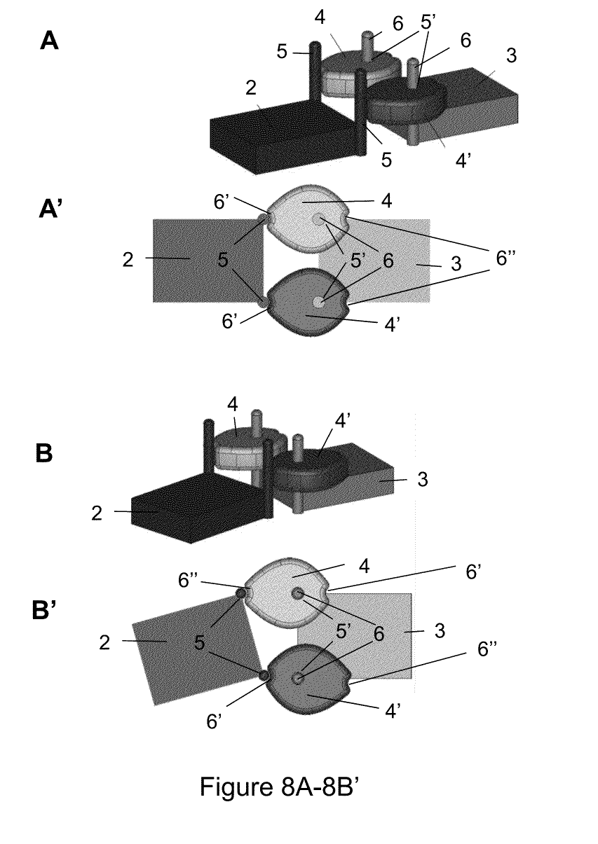

An example of adjustable alignment elements is shown in FIG. 8. The alignment elements (4, 4′) comprise at least three coupling features (5′, 6′, 6″): a hole (5′) and at least two notches (6′, 6″), which allow coupling of the alignment elements to elongated coupling features (5, 6) which are located on guiding elements (2, 3). When coupled to one elongated coupling feature only via the hole, the alignment element can rotate freely. When both alignment elements are coupled to two coupling features, thus via the hole and one of the notches (6′), the guiding elements are constrained to a certain relative position, as shown in FIG. 8A.

When one or more alignment elements are coupled to a coupling feature via another notch (6″), another relative position of the guiding elements is obtained, as shown in FIG. 8B.

### Example 9

### Alignment Elements Allowing Measurement of Distances or Forces

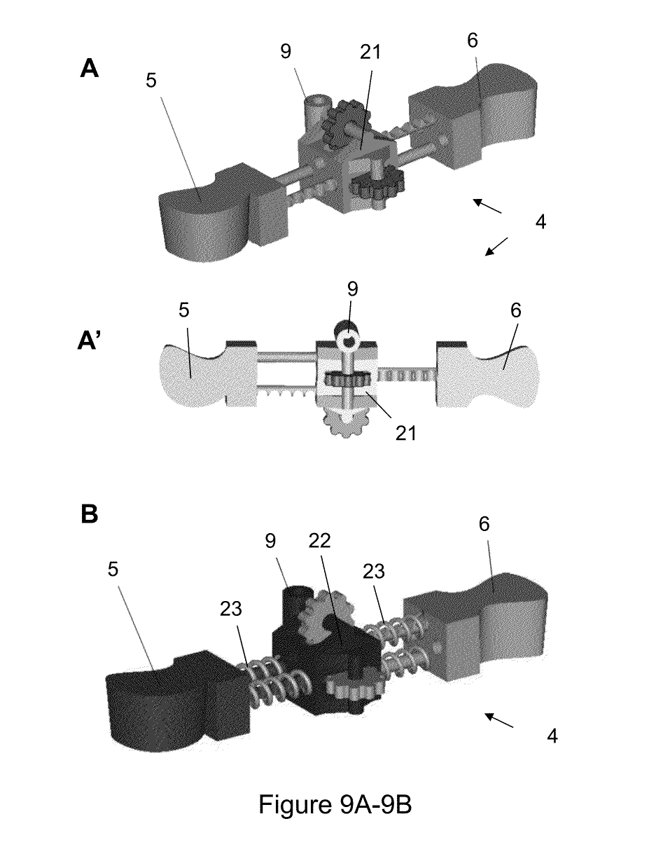

An example of an alignment element (4) comprising coupling features (5, 6) and a means (21) which allows the measurement of a distance is shown in FIGS. 9A and A′. The alignment elements may also contain further features such as a drill guide (9). An example of an alignment element (4) comprising coupling features (5, 6) and a means (22) which allows the measurement of a force is shown in FIG. 9B. For this purpose, the alignment element comprises springs (23). The alignment elements may also contain further features such as a drill guide (9). The alignment elements that allow measurement of distances or forces may also be adjustable. In this case, the alignment elements may be adjusted until a certain distance or force is obtained.

### Example 10

### Fixation of Guiding Elements

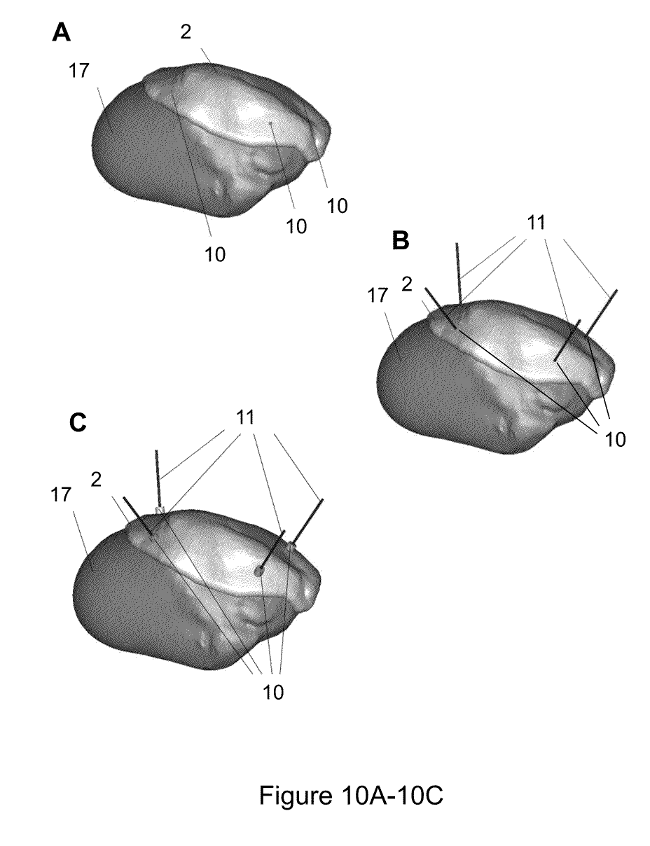

FIG. 10 shows how a guiding element can be fixed to an anatomical part using K-wires.

FIG. 10A shows a guiding element (2) comprising fixation features (10) positioned on an anatomical part (17) before fixation. The fixation features (10) are apertures in the guiding element (2), which can receive a K-wire. FIG. 10B shows the same guiding element, fixed on the anatomical part (17) by means of a K-wire (11), via the fixation features. FIG. 10C shows a guiding element, fixed on an anatomical part (17) by means of a K-wire (11), via cylindrical fixation features (10).

### Example 11

### Surgical Guide for the Repositioning of Two Anatomical Parts

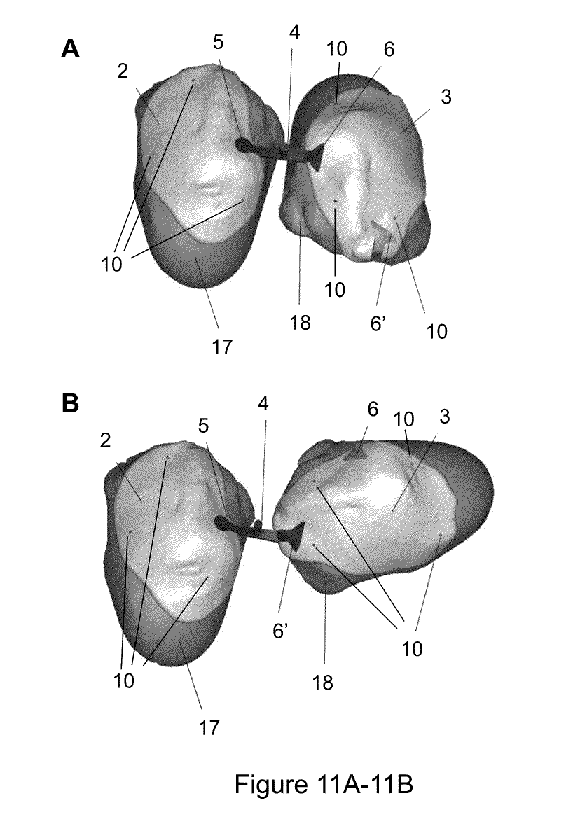

The surgical guides according to the present invention can be designed for the repositioning of two anatomical parts, as shown in FIG. 11. In this embodiment of the invention, each guiding element (2, 3) is positioned on a different anatomical part (17, 18). Both guiding elements comprise one or more coupling features (5, 6, 6′) which allow a mechanical constraint of the guiding elements by an alignment element (4) in a specific position. As illustrated in FIG. 11A, the alignment element (4) constrains the guiding elements in an initial position via coupling with two coupling features (5, 6) on the guiding elements.

As illustrated in FIG. 11B, the same alignment element (4) can constrain the guiding elements in a target position different from the initial position via coupling with another pair of coupling features (5, 6′) on the guiding elements.

### Example 12

### Surgical Guide for a Pelvic Bone

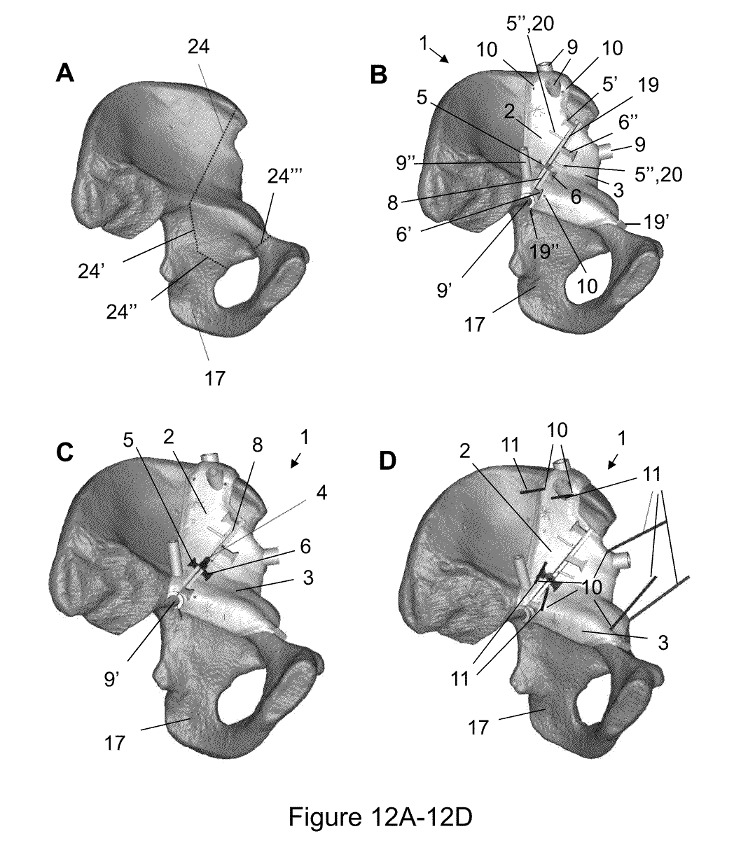

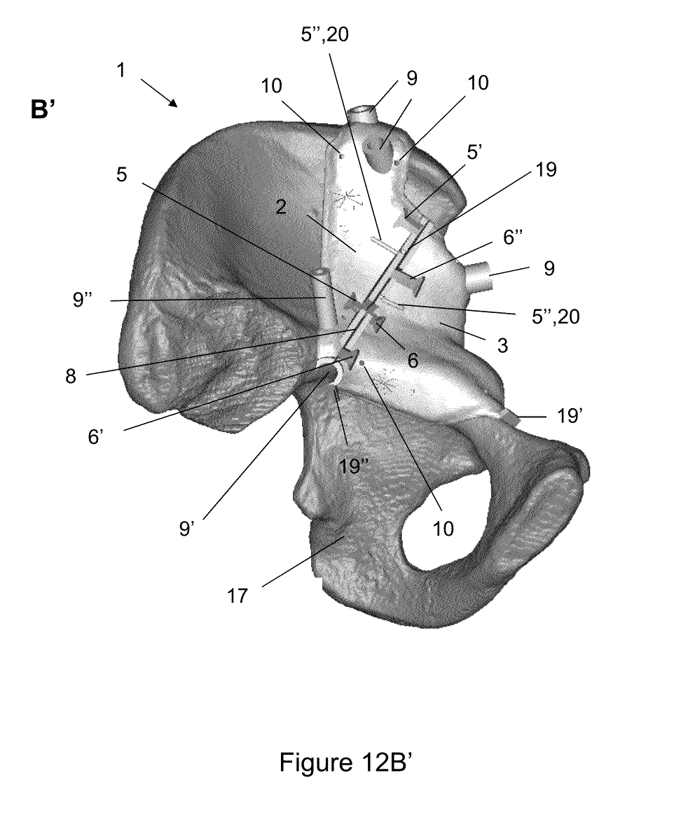

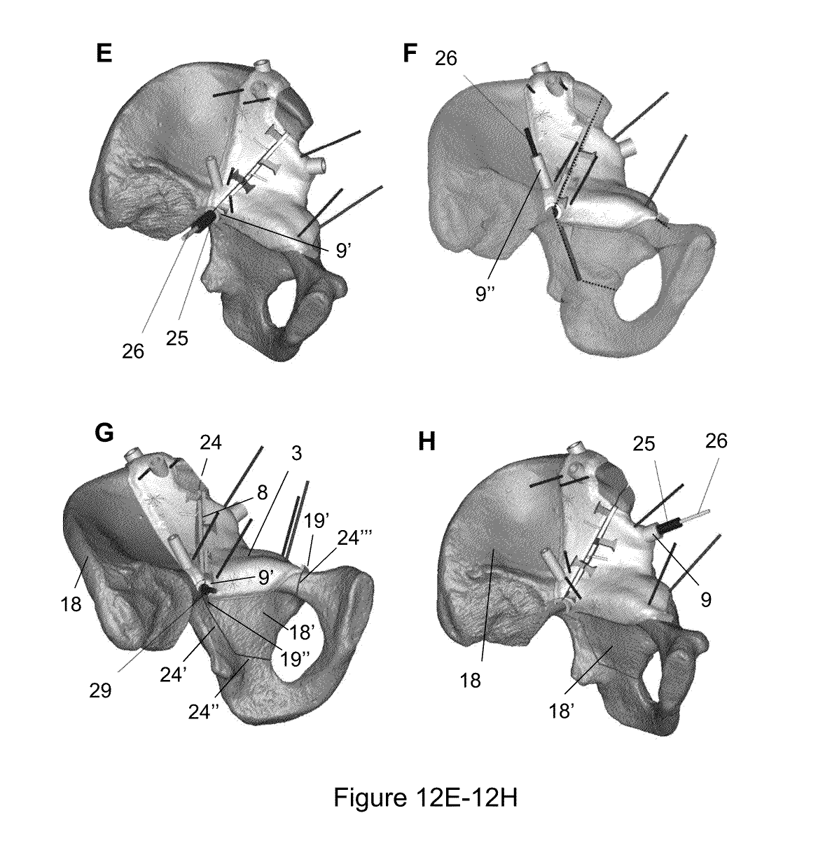

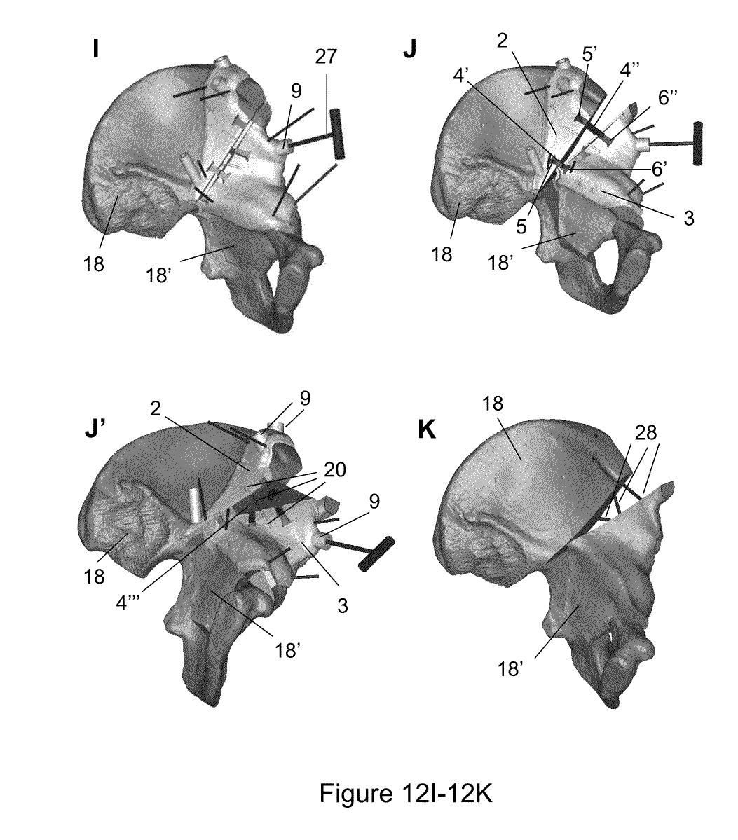

A surgical guide (1) was designed for guiding an osteotomy and repositioning of a pelvic bone. FIG. 12A shows a pelvic bone (17) and the locations (24, 24′, 24″, 24′″) where osteotomies are planned.

The surgical guide (1) comprises 2 guiding elements (2, 3). FIG. 12B shows the guiding elements (2, 3) when positioned on the pelvic bone (17). For clarity, FIG. 12B′ is an enlarged version of FIG. 12B. The guiding elements (2, 3) comprise coupling features (5, 6, 5′, 6′, 5″, 6″), drill guides (9, 9′, 9″) and fixation features (10). Some' of the coupling features (5″) are marks (20). The guiding elements (2, 3) further comprise one or more planar edges (19, 19′, 19″).

The surgical guide (1) further comprises a first alignment element (4), which also comprises coupling features (5, 6). Via coupling of the coupling features (5, 6) of the alignment element with coupling features (5, 6) on the guiding element, the alignment element (4) allows a mechanical constraint of the guiding elements in an initial position, as shown in FIG. 12C. In the initial position, the guiding elements (2, 3) form a cutting slot (8) and a drill guide (9′). The cutting slot (8) and drill guide (9′) are only provided when the guiding elements are constrained in the initial position. The drill guide (9′) is positioned at the intersection of two planned osteotomies (24, 24′).

FIG. 12D shows that when the initial position is obtained, the guiding elements (2, 3) can be fixed to the pelvic bone (17) via the fixation features (10), using K-wires (11). The fixation is not permanent, but removable.

In FIG. 12E, the drill guide (9′) guides a drill (26). The drilling occurs in the surface of one of the planned osteotomies (24), in order to weaken the bone. An insert (25) may be inserted into the drill guide (9′) to allow a better support of the drill (26).

In FIG. 12F, another drill guide (9″) guides a drill (26). Again, the drilling occurs in the surface of one of the planned osteotomies (24′), in order to weaken the bone. The drilling is stopped at the intersection of another one of the planned osteotomies (24″), thus avoiding excessive weakening of the bone. In the example shown in FIG. 12F, no insert is used.

Then, the osteotomies (24, 24′, 24″, 24′″) can be performed, as shown in FIG. 12G. The first osteotomy (24″) is not guided. The second osteotomy (24″′) is guided by the planar edge (19′) of guiding element (3). The third osteotomy (24) is guided by the cutting slot (8). The last osteotomy (24′) has been facilitated by the drilling as explained in FIG. 12F and is partially guided by a planar edge (19″) on guiding element (3). In order to recover an accurate corner between cutting slot (8) and planer edge (19″), (24, 24′), a protective filler (29) is inserted in drill guide (9′) before performing the osteotomies, as shown in FIG. 12G.

After performing the osteotomies, the pelvic bone is now cut into two parts (18, 18′).

In FIG. 12H, a drill guide (9) guides a drill (26), which is further stabilized by an insert (25) which is inserted in drill guide (9). After the drilling, the drill (26) and the insert (25) are removed. Then, a surgical instrument (27), specifically a repositioning instrument is inserted in the hole drilled as shown in FIG. 12I. The repositioning instrument allows for the repositioning of the bone part (18′), thus repositioning the acetabulum.

To find the correct relative positions of the bone parts (18, 18′) (target position), two alignment elements (4′, 4″) are coupled to the guiding elements (2, 3) via the coupling features (5, 6′, 5′, 6″) on the guiding elements, as shown in FIG. 12J. Alternatively, in this target position, the alignment element (4) as in the initial position could be used again, although in combination with different coupling features (5, 6′). The use of two alignment elements (4′, 4″) increases the accuracy of the repositioning.

In FIG. 12J′, the target position is achieved in an alternative way, by using an alignment element (4′″) which allows visual constraint of the guiding elements (2, 3). In the target position, the linear marks (20) on the guiding elements form a continuous line with the mark (20) on the alignment element (4″). Additionally, the shape of the alignment element (4′″) matches the surface of the osteotomy (24), if the bone parts (18, 18′) are in the target position. Additionally or alternatively, the alignment element (4′″) may contain a bone engagement surface and/or match the surface of one or both of the guiding elements.

When the target position is achieved, the bone parts (18, 18′) can be fixed by means of fixation pins (28). Insertion of the fixation pins can be facilitated by drilling holes, guided by the drill guides (9) on the guiding elements (2, 3). When the fixation pins are placed, the guiding elements are removed from the bone parts. The bone parts (18, 18′), fixed in the target position using fixation pins (28) are shown in FIG. 12K.

### Example 13

### Surgical Guide for a Pelvic Bone

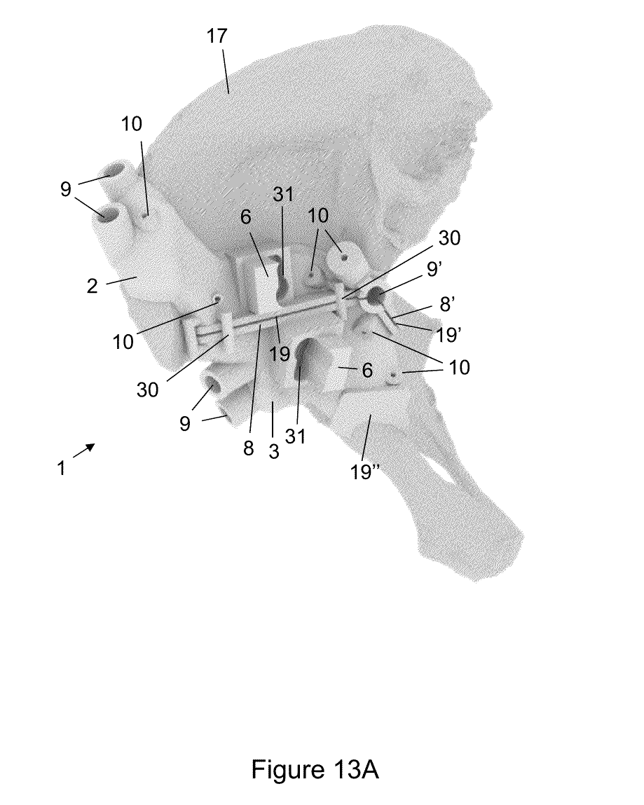

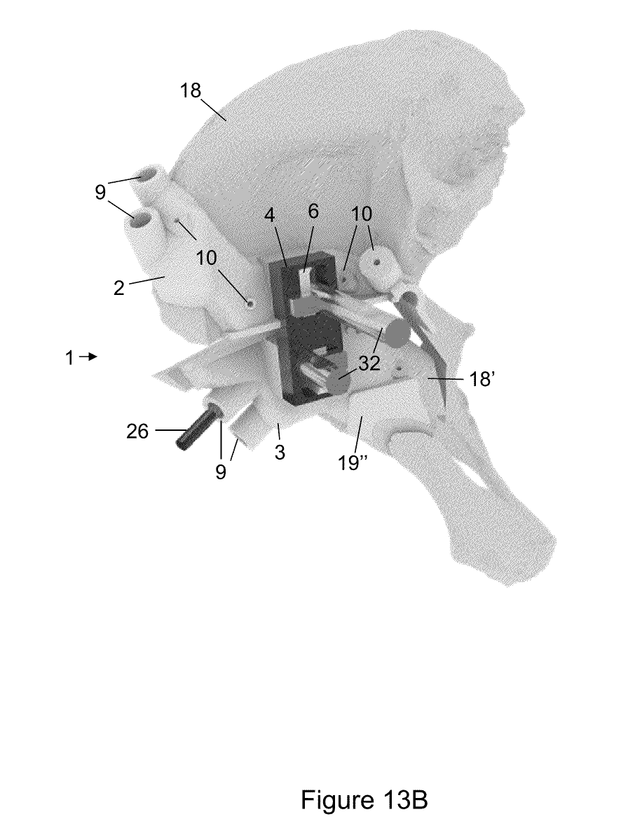

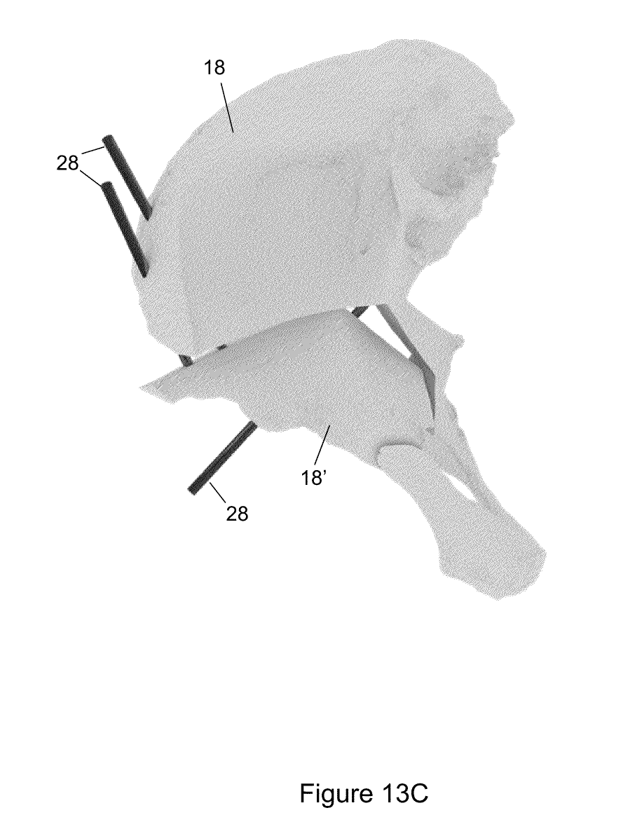

FIGS. 13A-C show the use of a particular embodiment of the guide (1) as described herein, for guiding an osteotomy and repositioning of a pelvic bone. The nature of the planned osteotomy and repositioning is similar as shown in Example 12 hereabove.

FIG. 13A shows the surgical guide (1) positioned in an initial position on a single pelvic bone (17). The surgical guide (1) comprises 2 guiding elements (2, 3), each comprising a coupling feature (6), fixation features (10), drill guides (9), an anatomy engagement surface (not shown) which anatomically matches a part of the pelvic bone (17). The guiding elements (2, 3) further comprise planar edges (19, 19′, 19″), some of which form cutting slots (8, 8′) in the initial position of the guiding elements on the pelvic bone.

The two guiding elements (2, 3) are manufactured as a single part. More particularly, the guiding elements (2, 3) are connected to each other in the initial position by two or more fixed connectors (30) which span a first cutting slot (8).

The guiding instrument (1) is designed for guiding an osteotomy and repositioning of the pelvic bone (17). More particularly, the anatomy engagement surfaces of the guiding elements (2, 3) allow for the positioning of the guiding instrument on the pelvic bone (17). The guiding elements (2, 3) are constrained in the initial position by the fixed connectors (30). Once positioned on the bone (17) according to surgical planning, the surgical guide can be fixed to the bone via the fixation features (10), using K-wires (not shown). The fixation is not permanent, but removable.

In the initial position, the guiding elements (2, 3) form a plurality of guiding features, more particularly two cutting slots (8, 8′) and a drill guide (9′). These guiding features are only provided when the guiding elements are constrained in the initial position. The drill guide (9′) is positioned at the intersection of the cutting slots (8, 8′). Accordingly, the drilling action guided by this drill guide (9′) weakens the bone along the intersection of the osteotomies guided by cutting slots (8, 8′) and thereby facilitates performing the osteotomies.

As in example 12, four osteotomies are performed, three of which are guided. Two of the osteotomies to be performed are guided by the cutting slots (8, 8′) which are formed in the initial position of the guiding elements (2, 3). In the osteotomy guided by the first cutting slot (8), the fixed connectors (30) between the guiding elements are cut. A third osteotomy is guided by a planar edge (19″) of the second guiding element (3). After performing the osteotomies, the pelvic bone is now cut into two parts (18, 18′).

The guide can be used for positioning the two bone parts (18, 18′) in a target position with the help of an alignment element (4), which comprises two coupling features (holes) which match the shape of the coupling features (6) of the guiding elements (2, 3). Via coupling of the coupling features of the alignment element with coupling features (5, 6) on the guiding element, the alignment element (4) allows a mechanical constraint of the guiding elements (and thereby also of the bone parts) in a target position, as shown in FIG. 13B.

In particular embodiments, the alignment element (4) can be reversibly locked to the guiding elements (2, 3) via locking keys (32). In such embodiments, the coupling features (6) of the guiding elements are typically provided with keyholes (30).

When the target position is achieved, the bone parts (18, 18′) can be fixed by means of fixation pins (28). Insertion of the fixation pins can be facilitated by drilling holes, guided by the drill guides (9) on the guiding elements (2, 3). When the fixation pins are placed, the guiding elements are removed from the bone parts. The bone parts (18, 18′), fixed in the target position using fixation pins (28) are shown in FIG. 13C.

1. A surgical guide system for two or more parts of a bone, comprising:
one or more independent alignment elements;
first and second patient-specific guiding elements, each containing one or more coupling features which allow a removable coupling of both said first and second guiding elements to said one or more alignment elements;
wherein said first guiding element has an anatomy engagement surface which anatomically matches part of the surface of a first of said two or more bone parts, and said second guiding element has an anatomy engagement surface which anatomically matches part of the surface of a second of said two or more bone parts; and
wherein the differing relative positions of the first and second patient-specific guiding elements define an initial and a target position and wherein a combination of at least one of said one or more alignment elements and at least two of said coupling features allows the coupling of said guiding elements to said one or more alignment elements in said target position.

2. The surgical guide system according to claim 1, wherein said guiding elements are coupled to each other in said initial position by one or more fixed connectors.

3. The surgical guide system according to claim 2, wherein said guiding elements are manufactured as a single part.

4. The surgical guide system according to claim 1, wherein, in said initial position, a guiding feature for a surgical tool is formed by the combination of two or more of said guiding elements.

5. The surgical guide system according to claim 4, wherein said guiding feature formed by the combination of two or more of said guiding elements is a cutting slot for an osteotomy.

6. The surgical guide system according to claim 1, wherein said coupling features comprise elements or combinations of elements selected from the group consisting of interlocking features, a snap-fit system, a dovetail system, a pinned system and a magnetic system.

7. The surgical guide system according to claim 1, wherein at least one of said coupling features allows visual alignment of said one or more alignment elements with one or more of said guiding elements.

8. The surgical guide system according to claim 1, wherein at least one of said alignment elements is patient-specific.

9. The surgical guide system according to claim 1, wherein at least one of said alignment elements contains an anatomy engagement surface which anatomically matches part of the surface of said anatomical part.

10. The surgical guide system according to claim 1, wherein at least one of said one or more coupling features is patient-specific and/or located on a patient-specific position on at least one of said guiding elements.

11. The surgical guide system according to claim 1, wherein at least one of the guiding elements and/or alignment elements further contains at least one element selected from the group consisting of a drill guide, a screw hole and a cutting slot.

12. The surgical guide system according to claim 11, wherein said drill guide contains a drill stop.

13. The surgical guide system according to claim 1, wherein at least one alignment element or coupling feature is adjustable.

14. The surgical guide system according to claim 1, wherein at least one of said alignment elements comprises fixation features, which allow for fixation of said alignment element to said anatomical part or parts.

15. The surgical guide system according to claim 1, wherein said anatomical part is a pelvic bone, or one or more vertebrae.

16. A method for generating a surgical guide system for two or more parts of a bone according to any of the claims above, which method comprises:
a) obtaining volume information of said bone;
b) planning a surgical operation or operations;
c) designing a surgical guiding system based on the information obtained in steps a) and b), said guiding system comprising:
at least one independent alignment element;
at least two patient-specific guiding elements, each having an anatomy engagement surface which anatomically matches part of the surface of one bone part, and each containing one or more coupling features which allows a removable coupling of an alignment element to said guiding element; wherein the fitting of said alignment element(s) in said coupling features allows the coupling of said guiding elements to said one or more alignment elements in a target position, and optionally an initial position, as determined by said surgical operation;
d) generating said at least one alignment element and said guiding elements based on step c.

17. The method of claim 16, wherein said method comprises producing said alignment element and/or said guiding elements by additive manufacturing.

18. A method for repositioning two or more bone parts of a bone, said method comprising:
a) fitting a separate guiding element comprising one or more coupling features which allow a removable coupling of said guiding element to one or more alignment elements on each of said two or more bone parts in an initial position,
b) (optionally simultaneously) fixing said guiding elements in an initial position by coupling at least one alignment element to said coupling features;
c) (optionally) fixing one or both of said guiding elements to the bone
d) performing a surgical operation on said bone, thereby optionally removing said at least one alignment element from said coupling features before or after performing said operation; and
e) fixing said guiding elements in a position different from the initial position by coupling at least one an alignment element to said coupling features.
